(19) United States
(12) Patent Application Publication
(10) Pub. No.: US 2008/0108147 A1
(43) Pub. Date: May 8, 2008

(54) Reduction of non-specific binding in immunoassays

(76) Inventors: Tie Wei, Bear, DE (US); Pratap Singh, Wilmington, DE (US); James Duffy, Landenberg, PA (US); Amy Posey, Cochranville, PA (US)

Correspondence Address: DADE BEHRING INC.;LEGAL DEPARTMENT, 1717 DEERFIELD ROAD, DEERFIELD, IL, 60015, US

(21) Appl. No.: 11/592,558

(22) Filed: Nov. 3, 2006

(51) Int. Cl. G01N 33/566; G01N 33/531; G01N 33/532; G01N 33/533; G01N 33/534; G01N 33/535; G01N 33/563

(52) U.S. Cl. 436/501; 436/547; 530/387.1

(57) ABSTRACT

Methods and compositions are disclosed for reducing non-specific binding in a binding assay for the determination of an analyte in a sample wherein one of the reagents for conducting the binding assay is an antibody reagent. The methods comprise treating the antibody reagent at a pH of about 2.0 to about 3.5. The method may further comprise treating the antibody reagent with a reducing agent in an amount sufficient to reduce non-specific binding of the antibody reagent. In some embodiments the reducing agent may be a thiol-containing reducing agent or a combination of two or more thiol-containing reducing agents.

### BACKGROUND OF THE INVENTION

In the fields of medicine and clinical chemistry, many studies and determinations of physiologically reactive species or analytes are carried out using conjugates involving specific binding pair members and supports and/or labels or the like. Various assay techniques that involve the binding of specific binding pair members are known. The analytes themselves are normally members of specific binding pairs, which allow for their detection employing a corresponding member of the specific binding pair to which the analyte in question belongs.

A variety of clinical conditions may be diagnosed and monitored by detecting the presence of and/or amount of a specific binding pair member in a sample. The results of chemical, biochemical, and biological assays are used to make important decisions; and, therefore, the accuracy and reliability of the result is of utmost importance. Heretofore, control samples of known concentration are assayed periodically, or even simultaneously with the sample to be measured, to calibrate and verify the operation of the assay on the unknown sample. This process reduces, but does not eliminate, the possibility of error in the assay of interest.

As the importance of measuring the presence of an analyte, which is a specific binding pair member, in a sample has increased, a number of means have been developed to detect such members. One method involves the conjugation of a label to a specific binding pair member that is employed as an assay reagent to bind to the analyte. In other approaches, a specific binding pair member for the detection of the analyte is conjugated to a support, which is employed as an assay reagent in various fashion along with other reagents to detect the analyte in question. Combinations of the above approaches are also utilized.

Assays in which a sample and one or more reagents are reacted in various ways to form a complex such as an antibody/antigen or similar complex, which may then be observed in order to measure the presence or level of an analyte or one or more of several analytes in the sample, are well known. Typically, in some embodiments an antibody is used to assay for the presence and/or amount of a hapten or an antigen for which the antibody is specific. The haptens and antigens include, for example, peptides, proteins, hormones, alkaloids, steroids, antibodies, nucleic acids, and fragments thereof, enzymes, cell surface receptors, and the like. As known in the art, heterogeneous assays are those in which one of the reactive binding partners is bound to a solid-phase. The various types of heterogeneous assays include, for example, the sandwich method, the indirect method, and the competitive method.

The usefulness of the assay reagent, however, will depend upon the specificity of the specific binding pair member for the other member, and also will depend upon the non-specific binding of the assay reagent. The non-specific binding often reduces the sensitivity of the heterogeneous assays. The degree of non-specific binding will limit the usefulness of the assay reagent. The greater the non-specific binding of the assay reagent, the lesser will be the sensitivity of the determination.

There remains a need for assay reagents, and antibody reagents in particular, which exhibit reduced non-specific binding when used in assays for the detection of one or more analytes.

### SUMMARY OF THE INVENTION

One embodiment of the present invention is a method for preparing an antibody reagent for use in an immunoassay. The method comprises treating the antibody reagent at a pH about 2.0 to about 3.5 to reduce non-specific binding of the antibody reagent. When the antibody reagent is unconjugated antibody, the above treatment is carried out in the absence of a chromatographic medium. In some embodiments the method may further comprise, either prior to or after the above treatment at low pH, treating the antibody reagent with a reducing agent in an amount sufficient to reduce non-specific binding of the antibody reagent. In some embodiments the antibody reagent may be an unconjugated antibody or in some embodiments the antibody reagent may be an antibody conjugated to a support, a member of a signal producing system or a member of a specific binding pair. In some embodiments the reducing agent may be a thiol-containing reducing agent or a combination of two or more thiol-containing reducing agents. In some embodiments, the reducing agent may be a borohydride or a phosphine such as, for example, sodium borohydride, tris(2-carboxyethyl) phosphine hydrochloride, and so forth.

Another embodiment of the present invention is a method for determining the presence and/or amount of an analyte in a sample suspected of containing the analyte. A sample and reagents for detecting the analyte are provided in combination. At least one of the reagents comprises an antibody reagent prepared according to one of the embodiments of the methods described above. The combination is incubated under conditions for binding of the analyte to one or more of the reagents. The presence and/or amount of binding of the analyte to one or more of the reagents are determined. The presence and/or amount of the binding are related to the presence and/or amount of the analyte in the sample. In some embodiments at least one other of the reagents for detecting the analyte comprises a second antibody specific for the analyte. In some embodiments the second antibody is pretreated either at a pH of about 2.0 to about 3.5 or with a reducing agent in an amount sufficient to reduce non-specific binding of the antibody reagent or a combination of the above pretreatment methods.

Another embodiment of the present invention is an antibody reagent prepared according to one of the embodiments of the methods described above.

### DESCRIPTION OF THE SPECIFIC EMBODIMENTS

Before describing the present invention in detail, it is to be understood that the terminology used herein is for the purpose of describing particular embodiments only and is not intended to be limiting. As used in this specification and the appended claims, the singular forms “a”, “an” and “the” include plural referents unless the content clearly dictates otherwise. As used herein, the phrase “at least” means that the indicated item is equal to or greater than that designated value and the term “about” means that the designated value may vary by plus or minus twelve percent, or eleven percent, or ten percent, or nine percent, or eight percent, or seven percent, or six percent, or five percent, or four percent, or three percent, or two percent, or one percent. The term “substantially” varies with the context as understood by those skilled in the relevant art and generally means at least 70%, preferably means at least 80%, more preferably at least 90%, and most preferably at least 95%. “Optionally” means that the specified item may be present or may not be present.

As mentioned above, in one aspect a method is provided for preparing an antibody reagent for use in an immunoassay. The method comprises treating the antibody reagent at a pH of about 2.0 to about 3.5 and/or with a reducing agent in an amount sufficient to reduce non-specific binding of the antibody reagent.

Non-specific binding, in general, means non-covalent binding between molecules or surfaces that is relatively independent of specific surface structures of the molecules or the surfaces. Non-specific binding is distinguished from specific binding, which involves the specific recognition of one of two different molecules for the other compared to substantially less recognition of other molecules. Non-specific binding may result from several factors including hydrophobic interactions between molecules, electrostatic or ion exchange interactions between molecules, contamination in an antibody reagent of molecules in ancillary reagents such as an ancillary enzyme substrate and signal producing system members and alike, species-specific interactions between molecules (e.g., human anti-mouse antibody, human anti-sheep antibody, human anti-bovine antibody, and the like), and so forth. The nature of the molecule or molecules that result in non-specific binding in assays is dependent on the nature of the sample, the assay milieu, the solid phase reagent surface, and so forth.

The sensitivity of the assay typically refers to the smallest mass of analyte that generates a statistically significant change in the signal generated by the assay when compared to the signal reading obtained in the absence of the analyte.

The binding assay generally involves specific binding between molecules. The molecules may be referred to as members of a specific binding pair (“sbp”), which means one of two different molecules, having an area on the surface or in a cavity, which specifically binds to and is thereby defined as complementary with a particular spatial and polar organization of the other molecule. The members of the specific binding pair may also be referred to as ligand and receptor (anti-ligand). These will usually be members of an immunological pair such as antigen-antibody, although other specific binding pairs such as biotin-avidin, hormones-hormone receptors, nucleic acid duplexes, IgG-protein A, polynucleotide pairs such as DNA-DNA, DNA-RNA, and the like are not immunological pairs but are included in the definition of sbp member. Binding assays are discussed in more detail below.

The reagents for conducting the binding assay usually include one or more sbp members such as, for example, antibodies, which may or may not be bound to other molecules depending on the nature of a particular assay in which the reagents are employed. One or more specific binding pairs may be utilized depending on the nature of the assay. The sbp member may or may not be bound to a support, a member of a signal producing system such as a label, an sbp member from a different specific binding pair, and so forth. Accordingly, the reagents for conducting an assay may include additional sbp members, ancillary reagents such as an ancillary enzyme substrate, signal producing system members, buffers, blocking agents for other forms of non-specific binding, and so forth. The reagents utilized for conducting a binding assay depend on the nature of the assay to be conducted and are discussed in detail below with respect to various assay embodiments. One or more reagents involving a solid phase or support such as, for example, a particle, may be employed in an assay depending on the nature of the assay.

One of the reagents for conducting a binding assay of interest with regard to the present methods is an antibody reagent. The term “antibody reagent” includes unconjugated antibody, antibody conjugated to a support, antibody conjugated to a member of a signal producing system such as a label, an antibody conjugated to a member of a specific binding pair, and so forth. “Unconjugated antibody” means an antibody that is not bound either covalently or non-covalently to another moiety. ‘Conjugated antibody” means an antibody that is bound either covalently or non-covalently to another moiety. For covalent bonding, the antibody may be bound through a bond or a linking group to another moiety to form a single structure. The antibody reagent is any reagent that is used in an assay for the determination of an analyte and that includes an antibody as all or part of the reagent; in many embodiments the antibody is specific for the analyte to be determined.

The term “antibody” means an immunoglobulin that specifically binds to and is thereby defined as complementary with a particular spatial and polar organization of another molecule. The antibody can be monoclonal or polyclonal and can be prepared by techniques that are well known in the art such as immunization of a host and collection of sera (polyclonal) or by preparing continuous hybridoma cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)₂, Fab′, and the like. In addition, aggregates, polymers, and conjugates of immunoglobulins or their fragments can be used where appropriate so long as binding affinity for a particular molecule is maintained.

As mentioned above, in some embodiments of the present methods, the antibody reagent may be treated at a pH of about 2.0 to about 3.5, or a pH of about 2.5 to about 3.5, or about 2.5 to about 3.0, or a pH of about 2.0 to about 2.5, or the like. Typically, a medium comprising the antibody reagent is treated to adjust the pH to within the above range. In accordance with this embodiment, a pH lowering agent is usually added to the medium since the pH of the medium is greater than the above range. The pH lowering agent may be an inorganic acid or an organic acid or a combination thereof. The pH lowering agent may be, for example, HCl, acetic acid, acetoacetic acid, ethylenediamine tetraacetic acid, formic acid, an amino acid, glycylglycine, isocitric acid, oxaloacetic acid, oxalic acid, phosphoric acid, phosphorous acid, pyruvic acid, succinic acid, tartaric acid, choloracetic acid, malic acid, citric acid, and the like, or a corresponding buffer composed of the salt/acid pair of one of the above acids, and so forth. The medium may comprise a buffering agent such as, for example, a buffer agent that can maintain pH between about 2 to about 3.5 including, but not limited to, those listed above, and the like.

The buffered medium solution may also contain a salt depending on the nature of the antibody reagent. Unconjugated antibody is usually present in a buffered solution as a soluble entity. In this embodiment, a salt concentration is utilized that is sufficient to maintain unconjugated antibody in a soluble form. The salt may be a chloride, bromide, fluoride, iodide, sulfate, or a combination thereof, and usually with a metal anion such as, for example, sodium, potassium, magnesium, manganese, cobalt, copper, and the like. The salt concentration depends on the nature of the salt, the nature of the antibody, the pH, reaction temperature, total ionic strength, and the like. Usually, the total salt concentration is about 0.5 M to about 3.0 M, about 1.0 M to about 3.0 M, about 1.0 M to about 2.0 M, 0.5 M to about 2.0 M, 1.0 M to about 2.5 M, and so forth. Examples of salts and their concentration, by way of illustration and not limitation, are 1 M sodium chloride and 0.1 M sodium citrate at pH 2.5, 2 M magnesium dichloride at pH 2.5, 1 M potassium chloride and 0.1 M potassium phosphate at pH 3.5, and so forth. In most embodiments, the salt concentration is substantially constant during the low pH treatment in accordance with the present methods. Although the above salt concentration has particular application to unconjugated antibody, low pH treatment of conjugated antibody optionally may be carried out using the above salt concentration parameters.

In addition to the above materials, the medium may also contain neutral or ionic detergents such as, for example, TWEEN 20®, TRITON® series (X100, X405 etc), ZWITTERGENT®, NP40®, Octylglucopyranoside, BRIJ® 35, CHAPS®, CHAPSO®, sodium dodecylsulfate (SDS), cholic acid, chremophor, taurocholic acid, GAFAC®, IGEPAL® and the like. In addition, the medium may also contain polyethyene glycol and chaotrops or chaotropic agents such as, for example, urea, guanidine hydrochloride, ammonium thiocyanate and the like. Polysaccharides such as, for example, dextran, and additional organic solvents such as dimethylsulfoxide (DMSO) and the like may also be present.

When the antibody reagent is unconjugated antibody, the low pH treatment in accordance with the present methods is carried out in the absence of a chromatographic medium although the treated antibody may be subsequently adjusted to a neutral pH (about 5 to about 8) prior to any necessary or desired chromatography purification using a neutral pH mobile phase. The pH may be adjusted to a neutral pH by addition of a suitable base or a suitable buffer or the like. The chromatographic medium referred to above includes silica-based chromatographic material, hydrophobic interaction chromatographic material, such as, for example, agarose-based chromatographic material, sepharose-based chromatographic material, and the like.

When the antibody reagent is a conjugated antibody and low pH treatment in accordance with the present methods is carried out, the treated conjugated antibody may be utilized after pH adjustment to a neutral pH as discussed above. Pretreatment of the antibody reagent at low pH as discussed above is carried out at a temperature of about 0° C. to about 70° C., or about 10° C. to about 70° C., about 20° C. to about 60° C., about 30° C. to about 50° C., about 35° C. to about 40° C., or the like. The time and temperature of the pretreatment depends on the nature of the antibody reagent, salt concentration, nature and concentration of the detergent present, the presence of other reagents such as chaotrops, the cause of non-specific binding, and so forth. The time of pretreatment is about 1 minute to about 72 hours, or about 10 minutes to about 24 hours, or about 10 minutes to about 8 hours, or about 20 minutes to about 6 hours, or about 30 minutes to about 4 hours, or about 45 minutes to about 3 hours, or about 1 hour to about 2 hours, and so forth.

Following the pretreatment of the antibody reagent at low pH as described above, the medium containing the antibody reagent may be adjusted to neutral pH, and/or separated from some specific components of the pretreatment medium. The approach utilized will depend on the nature of the components, and the like.

Prior to or after the above treatment of the antibody reagent at low pH as discussed above, the antibody reagent may be treated with a reducing agent. In either case, the medium containing the antibody reagent is treated, if necessary, to bring the pH into the range of about 5 to about 8. The reducing agent may be a sulfur-containing reducing agent such as a thiol-containing reducing agent, which includes, for example, 2-mercaptoethanol (2ME), dithiothreitol (DTT), dithioerythritol (DTE), cysteine, mercaptoacetic acid, and the like, or other reducing agents such as, for example, a borohydride, e.g., sodium borohydride and the like, or a phosphine, e.g., tris-(2-carboxyethyl) phosphine hydrochloride and the like, or bisulfite solutions especially a metabisulfite solution (MBS) or sodium bisulfite, or combinations thereof. The reducing agent is effective in achieving a reduction in non-specific binding for the antibody reagent in the absence of ethylene glycol or polyethylene glycol. Furthermore, although the medium with the reducing agent may contain a chelating agent, the concentration of the chelating agent is that which is only effective in preventing the formation of disulfide bonds; the concentration of the chelating is not such that the chelating agent contributes to rendering enhanced non-specific binding properties to the antibody reagent. The concentration of the chelating in the medium is discussed in more detail below.

The pretreatment of the antibody reagent is typically conducted in aqueous medium. The aqueous medium may be solely water or may include from about 0.01 to about 80 volume percent, and in some instances, about 0.1 to about 40 volume percent, of a cosolvent. The cosolvent may be an oxygenated hydrocarbon such as, for example, an alcohol, an ether, an amide, a ketone, a sulfoxide, and the like. Lower alkyl alcohols such as, for example, methanol, ethanol, propanol and so forth may be employed. When the method is not conducted at reduced pH, the pH for the medium is a moderate pH and is in the range of about 4 to about 11, or in the range of about 5 to about 10, or in the range of about 6.5 to about 9.5. Various buffers may be used to achieve the desired pH and maintain the pH during the pretreatment. Illustrative buffers include borate, phosphate, carbonate, tris, barbital and the like.

The amount of reducing agent employed is that which is effective in substantially reducing or eliminating non-specific binding when the antibody reagent is employed in the determination of an analyte. The phrase “substantially reducing” background interference, i.e., non-specific binding, means that the occurrence of non-specific binding is reduced by at least 20% relative to the incidence of non-specific binding occurring under the same set of conditions but without the use of the aforementioned pretreatment of the antibody reagent as provided herein. In some embodiments, non-specific binding is reduced by at least 30%, or by at least 40%, or by at least 50%, or by at least 60%, or by at least 70, or by at least 80%, or by at least 90%, or by at least 95%, or by at least 99%.

The concentration of the reducing agent is dependent on the nature of the reducing agent, the nature of the antibody reagent, the buffer medium, pH, temperature, the presence or absence of chelating agent, salt, detergent content, chaotrops, and the like. In some embodiments the molar content of the reducing agent(s) employed to effectively pretreat the antibody reagent is between about 0.00001 M and about 1.0 M, or between about 0.001 M and about 1.0 M, or between about 0.01 M and about 1.0 M, or between about 0.05 M and about 1.0 M, or between about 0.05 M and about 0.5 M, or between about 0.05 M and about 0.3 M, or between about 0.05 M and about 0.2 M, or the like.

As discussed above, the pretreatment solution may contain other substances such as, for example, a chelating agent, detergent, salt, chaotrops, and the like. The chelating agent is one that is effective in preventing or minimizing the formation of disulfide bonds. The chelating agent may be, for example, ethylene diamine tetraacetic acid or acetate (EDTA), EGTA, and the like. As explained above, the amount of chelating agent is that amount which is effective in protecting the reducing agent from degradation. Such amount (by weight) may be, for example, about 0.01 to about 1%, or about 0.05 to about 1%, or about 0.1 to about 1%, or about 0.1 to about 0.5%, and so forth.

Pretreatment of the antibody reagent with the reducing agent is carried out at a temperature of about 0° C. to about 100° C., or about 10° C. to about 80° C., about 20° C. to about 60° C., about 30° C. to about 50° C., about 35° C. to about 40° C., or the like. The time of the pretreatment depends on the nature of the reducing agent, the nature of the antibody reagent, presence or absence of chelating agent, detergent, salt, chaotrops, and so forth. The time of pretreatment with reducing agent is about 1 minute to about 72 hours, or about 10 minutes to about 24 hours, or about 10 minutes to about 8 hours, or about 20 minutes to about 6 hours, or about 30 minutes to about 4 hours, or about 45 minutes to about 3 hours, or about 1 hour to about 2 hours, and so forth.

In some embodiments two or more reducing agents may be employed in the pretreatment of the antibody reagent. In accordance with this embodiment the pretreatment solution may contain a mixture of two or more reducing agents, usually two reducing agents. This embodiment is particularly advantageous for thiol-containing reducing agents. The mixture may contain a ratio of one reducing agent to another reducing agent of about 95/5, about 90/10, about 85/15, about 80/20, about 75/25, about 70/30, about 65/35, about 60/40, about 55/45, about 50/50, about 45/55, about 40/60, about 35/65, about 30/70, about 25/75, about 20/80, about 15/85, about 10/90, about 5/95, and the like based upon weight. An example, by way of illustration and not limitation of a pretreatment reagent in accordance with this embodiment is one that contains between about 0.01 wt. % to about 1 wt. %, or about 0.1 wt. % to about 1 wt. %, of DTT and between about 0.01 wt. % to about 1 wt. %, or about 0.1 wt. % to about 1 wt. %, of 2ME in an aqueous pretreatment solution.

Following the pretreatment procedure, the reaction medium is treated to halt the effect of the reducing agent on the protein either by removing the reducing agent or rendering it inactive. In one approach the reaction medium is subjected to dialysis, chromatography, or the like to remove the reducing agent. The details of such separation techniques are well-known in the art and will not be repeated here.

In another approach a deactivation agent is added to the reaction medium in an amount effective to deactivate the reducing agent. When the reducing agent is a thiol-containing reducing agent, such deactivation agents include, for example, an oxidizing agent such as, e.g., copper sulfate, and the like. When the reducing agent is a borohydride, deactivation includes adjusting the pH to less than or equal to 6.0. In this approach the reaction medium is held for a time and at a temperature sufficient to deactivate the reducing agent. The time period and temperature, as well as other reaction parameters, are dependent on the nature of the deactivation agent, the presence or absence of a chelating agent, a detergent, a salt, chaotrops, and the like.

Following the pretreatment of the antibody reagent using a reducing agent as described above, the medium containing the antibody reagent is treated to remove excess reducing agent and/or side products. Such treatment may involve dialysis, diafiltration, chromatography or the like.

### Use of Pretreated Antibody Reagents

As discussed above, the antibody reagent treated in accordance with the above procedure can be used in a method for determining the presence and/or amount of an analyte in a sample suspected of containing the analyte. A combination is provided comprising the sample and reagents for detecting the analyte wherein at least one of the reagents comprises an antibody reagent prepared according to the above method. The combination is incubated under conditions for binding of the analyte to one or more of the reagents. The presence and/or amount of binding of the analyte to one or more of the reagents is determined wherein the presence and/or amount of the binding is related to the presence and/or amount of the analyte in the sample.

Accordingly, following the above pretreatment procedure, and depending on the nature of the antibody reagent, the pretreated antibody may be used in an assay for the determination of an analyte or may be employed to prepare one or more reagents for use in such an assay. Where the antibody reagent is an unconjugated antibody, the antibody may be employed in an assay as one of the reagents for detection of an analyte either in a single analyte assay or as one reagent in an assay for multiple analytes. Examples of various assay systems in which the pretreated antibody reagent may be employed are discussed in more detail hereinbelow.

On the other hand, a pretreated unconjugated antibody may be conjugated to another moiety such as, for example, a support, a member of a signal producing system, a member of a specific binding pair, and so forth. Usually, pretreated unconjugated antibody is used within a reasonable time period following the pretreatment procedure to conjugate it to another moiety. Accordingly, pretreated unconjugated antibody is employed within about 1 minute to about 2 years, or about 10 min to about 6 months, or the like of the pretreatment method or within the specified shelf life of the antibody reagent.

Where the antibody reagent is already a conjugated antibody, the pretreated material may be employed directly in an assay as one of the reagents for detection of an analyte either in a single analyte assay or as one reagent in an assay for multiple analytes. On the other hand, the pretreated conjugated antibody may be stored in accordance with known procedures for later use in an assay or for shipment to another location and/or entity.

As mentioned above, an antibody may be conjugated to a support. In general, the support is a solid phase, which is usually a porous or non-porous water insoluble material that can have any one of a number of shapes, such as a strip, a rod, a plate including planar plates, a well, a particle, a bead, and so forth. A wide variety of suitable supports are disclosed in Ullman, et al., U.S. Pat. No. 5,185,243, columns 10-11, which is incorporated herein by reference. The support may contain a plurality of molecules in the form of a microarray.

The surface of the support can be hydrophilic or capable of being rendered hydrophilic and includes inorganic powders such as chromium dioxide or other magnetic solid support, dendrimer, silica, magnesium sulfate, and alumina; natural polymeric materials, particularly cellulosic materials and materials derived from cellulose, such as fiber containing papers, e.g., filter paper, chromatographic paper, glass fiber paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly(4-methylbutene), polystyrene, polymethacrylate, poly(ethylene terephthalate), nylon, poly(vinyl butyrate), etc.; either used by themselves or in conjunction with other materials; glass such as, e.g., glass available as Bioglass, ceramics, metals, and the like. Natural or synthetic assemblies such as liposomes, phospholipid vesicles, and cells can also be employed. The support may include molded parts such as, for example, wells of a microtiter well plate, paddles, spheres, and so forth.

Particles may be uniform or non-uniform in shape and may be microscopic or macroscopic in size. The average diameter of the particles may be of at least about 20 nm and not more than about 20 microns, and in some instances, at least about 40 nm and less than about 10 microns, and in some instances at least about 0.3 microns to about 10 microns, and in some instances about 0.10 to 2.0 microns. The particle may have any density. In some embodiments the density of the particle approximates water, generally from about 0.7 to about 1.5 g/ml. The particles may or may not have a charge, and when they are charged, they are preferably negatively charged, although positively charged particles may be employed in some instances. The particles may be solid (e.g., comprised of organic and inorganic polymers or latex), oil droplets (e.g., hydrocarbon, fluorocarbon, silicon fluid), or vesicles (e.g., synthetic such as phospholipid or natural such as cells and organelles).

The particles can be biological materials such as cells and microorganisms, e.g., erythrocytes, leukocytes, lymphocytes, hybridomas, streptococcus, Staphylococcus aureus, E. coli, viruses, and the like. The particles can also be particles comprised of organic and inorganic polymers (either addition or condensation polymers), dendrimers, liposomes, latex particles, magnetic or non-magnetic materials, phospholipid vesicles, chylomicrons, lipoproteins, polysaccharide resin-based particles and the like. In some embodiments, the particles are a metal oxide such as, e.g., chromium dioxide particles (chrome particles), iron oxide particles, aluminum oxide particles, silicon dioxide particles, and the like. In some embodiments, the particles are quantum dots containing salts such as CdSe, ZnS, CdTe, MgS, MgSe, MgTe and the like. In some embodiments the particles are latex particles, latex particles impregnated with various organic dyes and complexes including but not limited to those of europium and dendrimers, or the like. Presence of these dyes and complexes thereof may allow generation of signal detected by fluorescence, chemiluminescence or electrochemiluminescence. The solid particles are usually readily dispersible in an assay medium.

The solid particles are also adsorptive or functionalizable so as to bind or attach at their surface, either directly or indirectly, another moiety such as, for example, an sbp member, label, coating such as, e.g., one or more polysaccharides, dendrimers, etc., or the like, and in some instances to incorporate within their volume a reactive reagent.

The solid particles can be comprised of polystyrene, polyacrylamide, homopolymers and copolymers of derivatives of acrylate and methacrylate, particularly esters and amides, silicones and the like. Oil droplets are water-immiscible fluid particles comprised of a lipophilic compound coated and stabilized with an emulsifier that is an amphiphilic molecule such as, for example, phospholipids, sphingomyelin, albumin and the like that exist as a suspension in an aqueous solution, i.e. an emulsion. Liposomes are microvesicles comprised of one or more lipid bilayers having approximately spherical shape and one of the preferred materials for use in the present invention.

Latex particles are a particulate water suspendable, water insoluble polymeric material usually having particle dimensions of 20 nm to about 2000 nm, in some instances about 100 to about 1000 nm in diameter. The latex may be a substituted polyethylene such as polyethylene glycol, polystyrene-butadiene, polyacrylamide polystyrene, polystyrene with amino groups, substituted poly-acrylic acid, substituted polymethacrylic acid, acrylonitrile-butadiene, styrene copolymers, polyvinyl acetate-acrylate, vinyl-chloride acrylate copolymers, and the like. Non-crosslinked polymers of styrene and carboxylated styrene or styrene functionalized with other active groups such as amino, hydroxyl, halo and the like are preferred. In some instances, copolymers of substituted styrenes with dienes such as butadiene may be used.

An antibody may be bound to a solid support in any manner known in the art, provided only that the binding does not substantially interfere with the ability of a binding partner for the antibody, such as an analyte, to bind with the antibody. In some embodiments, the antibody may be coated or covalently bound directly to the solid phase. The surface of the support may have layers of one or more carrier molecules such as poly(amino acids) including proteins such as serum albumins or immunoglobulins, or polysaccharides (carbohydrates) such as, for example, dextran or dextran derivatives. Linking groups may also be used to covalently couple the solid support and the antibody treated as described above. Other methods of binding an antibody to a support are also possible. For instance, a solid support may have a coating of a binder for a small molecule such as, for example, avidin, another antibody, etc., and a small molecule such as, e.g., biotin, hapten, etc., can be bound to pretreated antibody or vice versa. The binding of components to the surface of a support may be direct or indirect, covalent or non-covalent and can be accomplished by well-known techniques, commonly available in the literature. See, for example, “Immobilized Enzymes,” Ichiro Chibata, Halsted Press, New York (1978) and Cautrecasas, J. Biol. Chem., 245:3059 (1970).

The linking group may be a chain of from 1 to about 30 or more atoms, from about 1 to about 20 atoms, about 1 to about 10 atoms, each independently selected from the group normally consisting of carbon, oxygen, sulfur, nitrogen, and phosphorous, usually carbon and oxygen. The number of heteroatoms in the linking group normally ranges from about 0 to about 8, from about 1 to about 6, about 2 to about 4. The number of atoms in the chain is determined by counting the number of atoms other than hydrogen or other monovalent atoms along the shortest route between the substructures being connected. The atoms of the linking group may be substituted with atoms other than hydrogen such as carbon, oxygen and so forth in the form, e.g., of alkyl, aryl, aralkyl, hydroxyl, alkoxy, aryloxy, aralkoxy, and the like. As a general rule, the length of a particular linking group can be selected arbitrarily to provide for convenience of synthesis with the proviso that there be minimal interference caused by the linking group with the ability of the linked molecules to perform their function related to the assay in question.

Where a linking group is used to conjugate the antibody to the support or another moiety, the linking group may be aliphatic or aromatic. When heteroatoms are present, oxygen will normally be present as oxy or oxo, bonded to carbon, sulfur, nitrogen or phosphorous; sulfur will be present as thioether or thiono; nitrogen will normally be present as nitro, nitroso or amino, normally bonded to carbon, oxygen, sulfur or phosphorous; phosphorous will be bonded to carbon, sulfur, oxygen or nitrogen, usually as phosphonate and phosphate mono- or diester. Functionalities present in the linking group may include esters, thioesters, amides, thioamides, ethers, ureas, thioureas, guanidines, azo groups, thioethers, carboxylate and so forth. The linking group may also be a macro-molecule such as polysaccharides, peptides, proteins, nucleotides, and dendrimers.

Examples, by way of illustration and not limitation, of various linking groups that find use in the present invention are found in U.S. Pat. No. 3,817,837, particularly at column 30, line 69, to column 36, line 10, which disclosure is incorporated herein by reference in its entirety. Various linking groups and linking functionalities are disclosed in Cautrecasas, J. Biol. Chem. (1970) 245:3059. Examples of commercially available cross-linking reagents are disclosed in the Pierce Catalog and Handbook, Life Science and Analytical Research Products, Pierce Chemical Company, Rockford, Ill., 2005/2006.

Coated chromium dioxide particles are described in U.S. Pat. No. 4,661,408, issued to Lau, et al. on Apr. 28, 1987, the disclosure of which is hereby incorporated by reference. These chromium dioxide particles are sufficiently hydrolytically stable to be useful as solid supports in heterogeneous immunoassays and bioaffinity separations. The core of the particles is acicular, futile chromium dioxide having a surface area of 5-100 m²/g, coercivity of 100-750 oersteds, remnant magnetization of 5-45 emu/g and saturation magnetization of 8-85 emu/g (where emu is electromagnetic unit). These particles are surface stabilized and further stabilized with a coating of SiO₂. The silica coated chromium dioxide is then further coated with a silane to both further stabilize the particle and to provide binding sites for proteins. Antibody may be immobilized on chromium dioxide particles substantially according to the procedure described by Birkmeyer, et al., Clin. Chem. 33, 1543-1547 (1987), the disclosure of which is hereby incorporated by reference.

### Examples of Assays Employing Pretreated Antibody Reagents

As mentioned above, antibody reagents pretreated in accordance with the methods discussed above can be utilized in binding assays for analytes. The assay methods usually involve a sample suspected of containing an analyte, which is combined in an assay medium with reagents for carrying out the assay. Such reagents may include a support or solid phase that comprises an antibody, either an antibody pretreated as discussed above or a non-pretreated antibody. Other assay reagents can include a binding partner for the analyte (if the antibody on the solid support is not a binding partner for the analyte or if a sandwich assay is employed), analyte analogs, other solid supports to which one of the above reagents is bound, binding partners for sbp members, and so forth. One or more of the reagents may be part of a signal producing system where at least one of the reagents can be labeled; for example, an antibody reagent in accordance with the present methods may be an antibody conjugated to a label. The reagents are chosen such that a signal is obtained from a label in relation to the presence or amount of analyte in the sample. The assay can be performed either without separation (homogeneous) or with separation (heterogeneous) of any of the assay compounds or products. Where solid supports are utilized, the assay is usually heterogeneous although homogeneous formats using such reagents are known. The antibody reagent(s) pretreated in accordance with the methods described above have application to assays in which antibody reagents are employed such as, for example, all of the above assays.

Homogeneous immunoassays are exemplified by the EMIT® assay products (Dade Behring Inc., Newark Del.) disclosed in Rubenstein, et al., U.S. Pat. No. 3,817,837, column 3, line 6 to column 6, line 64; enzyme channeling techniques such as those disclosed in Maggio, et al., U.S. Pat. No. 4,233,402, column 6, line 25 to column 9, line 63; and other enzyme immunoassays such as the enzyme linked immunosorbant assay (“ELISA”) are discussed in Maggio, E. T., infra. The above disclosures are all incorporated herein by reference.

Heterogeneous assays usually involve one or more separation steps and can be competitive or non-competitive. A variety of competitive and non-competitive heterogeneous assay formats are disclosed in Davalian, et al., U.S. Pat. No. 5,089,390, column 14, line 25 to column 15, line 9, incorporated herein by reference. In a typical competitive heterogeneous assay, a support having an antibody for analyte bound thereto is contacted with a medium containing the sample and analyte analog conjugated to a detectable label such as an enzyme (the “enzyme conjugate”). Analyte in the sample competes with the enzyme conjugate for binding to the antibody. After separating the support and the medium, the label activity of the support or the medium is determined by conventional techniques and is related to the amount of analyte in the sample.

Another example of an assay is a combination of a homogeneous and heterogeneous assay format as disclosed in Singh, et al., U.S. Pat. No. 6,083,708 and in Clin. Chem. 40, 1845-1849 (1994). In this format, analyte-specific antibody is covalently coupled onto a Starburst™ Dendrimer, a water-soluble highly functionalized polymer of controlled architecture and defined molecular weight. A clear aqueous solution of the antibody-dendrimer complex is contacted with a medium containing the analyte of interest. The antibody-dendrimer-analyte complex is adsorbed on a solid phase with a negative charge such as glass fiber filter paper. After a wash and separation step, the solid support is contacted with a medium containing the second analyte-specific antibody, which contains an enzyme label such as alkaline phosphatase. The support is contacted with a medium that contains a fluorometric substrate for the enzyme and also separates the non-specifically bound species present along with the analyte. The presence and amount of the enzyme label present on the support is related to the presence and amount of the analyte in the medium. The above assay may be carried out using the STRATUS® CS analyzer from Dade Behring Inc., Newark, Del. For such a system, the antibody utilized for coupling onto dendrimer may be pretreated in accordance with embodiments of the invention discussed above.

A typical non-competitive sandwich assay is an assay disclosed in David, et al., U.S. Pat. No. 4,486,530, column 8, line 6 to column 15, line 63, incorporated herein by reference. In this method, an immune sandwich complex is formed in an assay medium. The complex comprises the analyte, a first antibody (monoclonal or polyclonal) that binds to the analyte and a second antibody that binds to the analyte or a complex of the analyte and the first antibody. Subsequently, the immune sandwich complex is detected and is related to the amount of analyte in the sample. The immune sandwich complex is detected by virtue of the presence in the complex of a label wherein either, or both, the first antibody and the second antibody contain labels or substituents capable of combining with labels.

Sandwich assays find use for the most part in the detection of antigen and receptor analytes. In the assay the analyte is bound by two antibodies specific for the analyte and, thus, the assay is also referred to as the two-site immunometric assay. In one approach a first incubation of unlabeled antibody coupled to a support, otherwise known as the immobilized antibody, is contacted with a medium containing a sample suspected of containing the analyte. After a wash and separation step, the support is contacted with a medium containing the second antibody, which generally contains a label, for a second incubation period. The support is again washed and separated from the medium and either the medium or the support is examined for the presence of label. The presence and amount of label is related to the presence or amount of the analyte. For a more detailed discussion of this approach see U.S. Pat. Nos. Re 29,169 and 4,474,878, the relevant disclosures of which are incorporated herein by reference.

In a variation of the above sandwich assay the sample in a suitable medium is contacted with labeled antibody for the analyte and incubated for a period of time. Then, the medium is contacted with a support to which is bound a second antibody for the analyte. After an incubation period, the support is separated from the medium and washed to remove unbound reagents. The support or the medium is examined for the presence of the label, which is related to the presence or amount of analyte. For a more detailed discussion of this approach see U.S. Pat. No. 4,098,876, the relevant disclosure of which is incorporated herein by reference.

In another variation of the above, the sample, the first antibody bound to a support and the labeled antibody are combined in a medium and incubated in a single incubation step. Separation, wash steps and examination for label are as described above. For a more detailed discussion of this approach see U.S. Pat. No. 4,244,940, the relevant disclosure of which is incorporated herein by reference.

A particular example of an assay is described below by way of illustration and not limitation. Such assay is referred to as an induced luminescence immunoassay and is described in U.S. Pat. No. 5,340,716 (Ullman, et al.), which disclosure is incorporated herein by reference. In one approach the assay uses a particle incorporating a photosensitizer and a label particle incorporating a chemiluminescent compound. The label particle is conjugated to an sbp member that is capable of binding to an analyte to form a complex, or to a second sbp member to form a complex, in relation to the presence of the analyte. If the analyte is present, the photosensitizer and the chemiluminescent compound come into close proximity. The photosensitizer generates singlet oxygen and activates the chemiluminescent compound when the two labels are in close proximity. The activated chemiluminescent compound subsequently produces light. The amount of light produced is related to the amount of the complex formed, which in turn is related to the amount of analyte present.

By way of further illustration, a chemiluminescent particle is employed, which comprises the chemiluminescent compound associated therewith such as by incorporation therein or attachment thereto. An sbp member that binds to the analyte is bound to these particles. A second sbp member that binds to the analyte is part of a biotin conjugate. Streptavidin is conjugated to a second set of particles (photosensitizer particles) having a photosensitizer associated therewith. The chemiluminescent particles are combined in a reaction medium with a sample suspected of containing an analyte and the photosensitizer particles. The reaction medium is incubated to allow the particles to bind to the analyte by virtue of the binding of the sbp members to the analyte. Then, the medium is irradiated with light to excite the photosensitizer, which is capable in its excited state of activating oxygen to a singlet state. Because the chemiluminescent compound of one of the sets of particles is now in close proximity to the photosensitizer by virtue of the presence of the analyte, it is activated by the singlet oxygen and emits luminescence. The medium is then examined for the presence and/or the amount of luminescence or light emitted, the presence thereof being related to the presence of the analyte.

Another particular example of an assay to which the present soluble conjugates have application is discussed in U.S. Pat. No. 5,616,719 (Davalian, et al.), which describes fluorescent oxygen channeling immunoassays.

The homogeneous or heterogeneous assays discussed above are normally carried out in an aqueous buffered medium at a moderate pH, generally that which provides optimum assay sensitivity. The aqueous medium may be solely water or may include from about 0.1 to about 80 volume percent, from about 0.1 to about 40 volume percent, of a cosolvent. The pH for the medium will usually be in the range of about 4 to about 11, more usually in the range of about 5 to about 10, and preferably in the range of about 6.5 to about 9.5. The pH will usually be a compromise between optimum binding of the binding members of any specific binding pairs, the pH optimum for other reagents of the assay such as members of the signal producing system, and so forth.

Various buffers may be used to achieve the desired pH and maintain the pH during the determination. Illustrative buffers include borate, phosphate, carbonate, tris, barbital and the like. The particular buffer employed is not critical to this invention, but in an individual assay one or another buffer may be preferred. Various ancillary materials may be employed in the above methods. For example, in addition to buffers the medium may comprise stabilizers for the medium and for the reagents employed. Frequently, in addition to these additives, proteins may be included, such as albumins; organic solvents such as formamide; quaternary ammonium salts; polyanions such as dextran sulfate; surfactants, particularly non-ionic surfactants; binding enhancers, e.g., polyalkylene glycols; or the like.

One or more incubation periods may be applied to the medium at one or more intervals including any intervals between additions of various reagents mentioned above. The medium is usually incubated at a temperature and for a time sufficient for binding of various components of the reagents to occur. Moderate temperatures are normally employed for carrying out the method and usually constant, controlled temperatures are preferred during the period of the measurement. Incubation temperatures normally range from about 5° to about 70° C., usually from about 15° C. to about 70° C., more usually 20° C. to about 45° C. The time period for the incubation is about 0.2 seconds to about 6 hours, usually, from about 2 seconds to about 1 hour, more usually, about 1 to about 5 minutes. The time period depends on the temperature of the medium and the rate of binding of the various reagents, which is determined by the association rate constant, the concentration, the binding constant and dissociation rate constant. Temperatures during measurements will generally range from about 10 to about 50° C., more usually from about 15 to about 40° C.

The concentration of analyte that may be assayed generally varies from about 10⁻⁵ to about 10⁻¹⁷ M, more usually from about 10⁻⁶ to about 10⁻¹⁴ M. Considerations, such as whether the assay is qualitative, semi-quantitative or quantitative (relative to the amount of analyte present in the sample), the particular detection technique and the concentration of the analyte normally determine the concentrations of the various reagents.

The concentration range of interest of the analyte will generally determine the concentrations of the various reagents in the assay medium. However, the final concentration of each of the reagents is normally determined empirically to optimize the sensitivity of the assay over the range. That is, a variation in concentration of analyte that is of significance should provide an accurately measurable signal difference. Considerations such as the nature of the signal producing system and the nature of the analytes normally determine the concentrations of the various reagents.

While the order of addition may be varied widely, there will be certain preferences depending on the nature of the assay. The simplest order of addition is to add all the materials simultaneously and determine the effect that the assay medium has on the signal as in a homogeneous assay. Alternatively, the reagents can be combined sequentially. Optionally, an incubation step may be involved subsequent to each addition as discussed above.

The following examples further describe the specific embodiments of the invention by way of illustration and not limitation and are intended to describe and not to limit the scope of the invention.

In a homogeneous assay after all of the reagents have been combined, the signal is determined and related to the amount of analyte in the sample. For example, in an EMIT assay, a sample suspected of containing an analyte is combined in an aqueous medium either simultaneously or sequentially with an enzyme conjugate of an analyte analog and with antibody capable of recognizing the analyte. Generally, a substrate for the enzyme is added, which results in the formation of a chromogenic or fluorogenic product upon enzyme catalyzed reaction. Preferred enzymes are glucose-6-phosphate dehydrogenase and alkaline phosphatase but other enzymes may be employed. The analytes and the moieties of the enzyme conjugate compete for binding sites on the antibody. The enzyme activity in the medium is then determined, usually by spectrophotometric means, and is compared to the enzyme activity determined when calibrators or reference samples are tested in which a known amount of the analytes is present. Typically, the calibrators are tested in a manner similar to the testing of the sample suspected of containing the analytes. The calibrators typically contain differing, but known, concentrations of the analyte to be determined. Preferably, the concentration ranges present in the calibrators span the range of suspected analyte concentrations in the unknown samples.

The aforementioned assays may be carried out using mutant glucose-6-phosphate dehydrogenase as the enzyme of the enzyme conjugate. This mutant enzyme is described in U.S. Pat. Nos. 6,090,567 and 6,033,890, the relevant disclosures of which are incorporated herein by reference.

As discussed above, heterogeneous assays usually involve one or more separation steps and can be competitive or non-competitive. In one type of competitive assay using reagents in accordance with embodiments of the present invention, a support, as discussed above, having antibodies for an analyte bound thereto, where the support with bound antibody has been pretreated in accordance with the present methods, is contacted with a medium containing the sample and appropriate enzyme conjugates. After separating the support and the medium, the enzyme activity of the support or the medium is determined by conventional techniques and related to the presence and/or amount of the analyte in the sample.

Activation of a signal producing system depends on the nature of the signal producing system members. For those members of a signal producing system that are activated with light, the member is irradiated with light. Other activation methods will be suggested to those skilled in the art in view of the disclosures herein. For some signal producing systems, no agent for activation is necessary such as those systems that involve a label that is a radioactive label, an enzyme, and so forth. For enzyme systems addition of a substrate and/or a cofactor may be necessary.

In certain embodiments a second enzyme may be employed in addition to the enzyme of the enzyme conjugate. The enzymes of the pair of enzymes are related in that a product of the first enzyme serves as a substrate for the second enzyme.

The examination for presence and amount of the signal also includes the detection of the signal, which is generally merely a step in which the signal is read. The signal is normally read using an instrument, the nature of which depends on the nature of the signal. The instrument may be a spectrophotometer, fluorometer, absorption spectrometer, luminometer, chemiluminometer, actinometer, photographic instrument, and the like. The presence and amount of signal detected is related to the presence and amount of the analyte present in a sample. Temperatures during measurements generally range from about 10° to about 70° C., more usually from about 20° to about 45° C., more usually about 20° to about 25° C. In one approach standard curves are formed using known concentrations of the analytes to be screened. As discussed above, calibrators and other controls may also be used.

Another embodiment of an assay format is a capture assay. In this assay format, the antibody for the analyte is covalently bound to a magnetic particle such as, for example, a chromium dioxide particle, or to a non-magnetic particle such as, for example, polystyrene beads. The sample is incubated with these particles to allow the analyte in the sample to bind to the antibodies. Subsequently, an enzyme that has the analyte or analyte analog covalently attached is incubated with the magnetic particles. After washing, the amount of enzyme that is bound to the magnetic particles is measured and is inversely related to the presence and/or amount of the analyte in the sample.

The following specific assay descriptions are by way of illustration and not limitation on the scope of the present invention. Selection of a particular analyte in the following descriptions is also by way of illustration and not limitation as the present invention has general application to detection of any type of analyte as discussed above.

In one embodiment, rapamycin-label compounds can be used in an immunoassay or receptor based assay as the first part of the detection molecule by mixing the test sample or a rapamycin standard with a rapamycin-oxime conjugate such as a biotin ester of rapamycin and allowing them to compete for binding to an antibody for rapamycin, which antibody has been pretreated in accordance one embodiment of the present methods. After rinsing with an appropriate wash buffer, a detection molecule consisting of streptavidin or avidin conjugated to an enzyme, fluorescent or chemiluminescent molecule or radioactive moiety can be used.

In one embodiment the assay is an induced luminescence assay as described above. The reagents include two latex bead reagents and a biotinylated anti-rapamycin mouse monoclonal antibody. This antibody conjugate is pretreated in accordance with an embodiment of one of the present methods. The first bead reagent is coated with rapamycin or a rapamycin analog and contains chemiluminescent dye. The second bead reagent is coated with streptavidin and contains a photosensitizer dye. In a first step, sample is incubated with biotinylated antibody, which allows rapamycin from the sample to saturate a fraction of the biotinylated antibody that is directly related to the rapamycin concentration. In a second step, the first bead reagent is added and leads to the formation of bead/biotinylated antibody immunocomplexes with the non-saturated fraction of the biotinylated antibody. The second bead reagent is then added and binds to the biotin to form bead pair immunocomplexes. When illuminated by light at 680 nm, the second bead reagent converts dissolved oxygen in the reaction solution into the more energetic singlet oxygen form. In the bead pairs, the singlet oxygen diffuses into the first bead reagent thereby triggering a chemiluminescent reaction. The resulting chemiluminescent signal is measured at 612 nm and is an inverse function of the concentration of rapamycin in the sample. The amount of this signal is related to the presence of amount of rapamycin in the sample.

Another example of an assay is a solid phase enzyme immunoassay intended to quantitatively measure the N-terminal pro-brain natriuretic peptide (NT pro-BNP) in human serum and plasma for monitoring congestive heart failure. In the assay, patient sample is mixed with chromium dioxide particles coated with antibodies specific for NT-proBNP. The antibody-coated particles are pretreated in accordance with one of the above embodiments in accordance with the present methods. Another reagent is an enzyme conjugate reagent (alkaline phosphatase labeled antibody specific for NT-proBNP). A particle/NT-prQBNP/conjugate sandwich forms during the incubation period. Unbound conjugate is washed away and the remaining chromium dioxide particles carrying the immuno-sandwich are transferred into an assay test container containing an enzyme amplification cascade system as described by Harbron, et al. Analytical Biochemistry 206, 119-124 (1992). In this system the cascade detects alkaline phosphatase via enzymatic hydrolysis of the substrate FADP to produce the coenzyme FAD. This coenzyme activates D-Amino acid oxidase and the activated holo D-Amino acid oxidase oxidizes D-proline to produce hydrogen peroxide. The amount of hydrogen peroxide produced is quantitated by the hydrogen peroxidase-mediated reaction of hydrogen peroxide with 3,5-dichloro-2-hydroxybenzenesulfonic acid and 4-aminoantipyrine to produce a colored product measured at 510 nm spectrophotometrically. The absorption at 510 nm is directly proportional to the NTproBNP concentration in the patient sample reported in units of pg/mL or ng/dL. The above assay may be carried out using the Dimension® analyzer from Dade Behring Inc., Newark, Del. The antibody utilized for coating chromium dioxide particles is pretreated in accordance with embodiments of the invention discussed above. In addition, the antibody-coated chromium dioxide particles are treated in accordance with embodiments of the invention discussed above.

In another example, free thyroxine, FT4, is a thyroid function test that measures the fraction of T4 that is not protein bound but is physiologically available. Such an assay employs chromium dioxide particles with immobilized anti-T4 antibody as a separation solid phase. The anti-T4 antibody is pretreated in accordance with one of the embodiments of the present methods discussed above. A thyronine-alkaline phosphatase conjugate (T3-ALP) is provided as FT4 conjugate reagent. During the assay, FT4 from a sample to be analyzed is incubated with anti-FT4 antibody coupled chromium dioxide particle based reagent and the T3-ALP conjugate. A low level of FT4 present in the mixture allows a high level of the T3-ALP to bind with the chromium dioxide particles, resulting in high enzyme level. Conversely, a high level of FT4 would allow a low level of the conjugate to bind with the chromium dioxide particles and resulting in low enzyme level. The level of enzyme may be detected via a cascade based signal generation process described above. The result is a negative calibration curve, which is fit by non-linear regression using the logit model and then used to compute final test results in analyte units of ng/dL or pmol/L.

A specific example of another assay format is ACMIA (Affinity Column Mediated Immuno Assay). For the ACMIA assay format for a drug such as rapamycin, chrome particles, which are coated with rapamycin or a rapamycin analog, are employed as a first component. A second component is an antibody for rapamycin that is covalently linked to a reporter enzyme (usually beta-galactosidase). This reagent is added to a reaction vessel in excess. Either the free antibody or the antibody conjugate may be pretreated in accordance with one embodiment of the present methods. The antibody-enzyme conjugate is mixed with a sample to allow the analyte to bind to the antibody. Next, the chrome reagent is added to bind up any excess antibody-enzyme conjugate. Then, a magnet is applied, which pulls all of the chrome and excess antibody-enzyme out of the suspension, and the supernatant is transferred to a final reaction container. The substrate of the reporter enzyme is added to the final reaction container, and enzyme activity is measured spectrophotometrically as a change in absorbance over time. The antibody for rapamycin as part of the second component is in accordance with embodiments of the invention discussed above. The amount of this signal is related to the presence of amount of rapamycin in the sample.

In a sandwich assay format, a first reagent comprising chrome particles coated with anti-NT pro-BNP antibodies (or NT pro-BNP binding partner), and a second reagent comprising a second antibody (or binding partner) conjugated to a reporter enzyme are employed. In this format, the sample is incubated with the chrome particles so that all of the NT pro-BNP in the sample becomes bound to the chrome particles. The chrome particles are washed, using a magnet to separate the bound analyte from the supernatant. Then, the second reagent, i.e., antibody (or binding partner) conjugated to a reporter enzyme, is incubated with the chrome particles to form a “sandwich”. After washing, the amount of enzyme that is bound to the chrome is measured and is related to the presence and/or amount of NT pro-BNP in the sample. At least one of the antibodies of the first and second antibody reagents is in accordance with embodiments of the invention discussed above.

Another assay format is EMIT (Enzyme-Mediated Immunoassay Technology). In this assay format, an enzyme conjugate is formed such as, for example, a conjugate of G-6-PDH and an analyte such as Sirolimus. An antibody for Sirolimus is incubated with the enzyme-conjugate and a sample suspected of containing Sirolimus. Antibody for Sirolimus binds to the Sirolimus analyte in the sample instead of binding to the enzyme conjugate, which reduces the amount of inhibition of the enzyme activity that might otherwise occur in the absence of Sirolimus in the sample. In this way, samples with more analyte will yield higher enzyme activity, and samples with no analyte will have the maximum inhibition and the lowest enzyme activity. The amount of reduction of inhibition of enzyme activity is related to the amount of Sirolimus in the sample. At least the antibody for Sirolimus is pretreated in accordance with embodiments of the invention discussed above.

### Discussion of Terms

Before proceeding further with the description of examples of specific embodiments of the aforementioned materials and methods, a number of terms employed above will be defined.

Analyte—the compound or composition to be detected. The analyte can be comprised of a member of a specific binding pair (sbp) and may be a ligand, which is usually monovalent (monoepitopic), usually haptenic, and is a single compound or plurality of compounds which share at least one common epitopic or determinant site.

The monoepitopic ligand analytes will generally be from about 100 to 2,000 molecular weight, more usually from 125 to 1,000 molecular weight. The analytes include drugs, metabolites, pesticides, pollutants, and the like. Representative analytes, by way of example and not limitation, include (i) alkaloids such as morphine alkaloids, which include morphine, codeine, heroin, dextromethorphan, their derivatives and metabolites; cocaine alkaloids, which include cocaine and benzyl ecgonine, their derivatives and metabolites; ergot alkaloids, which include the diethylamide of lysergic acid; steroid alkaloids; iminazoyl alkaloids; quinazoline alkaloids; isoquinoline alkaloids; quinoline alkaloids, which include quinine and quinidine; diterpene alkaloids, their derivatives and metabolites; (ii) steroids, which include the estrogens, androgens, andreocortical steroids, bile acids, cardiotonic glycosides and aglycones, which includes digoxin and digoxigenin, saponins and sapogenins, their derivatives and metabolites; steroid mimetic substances, such as diethylstilbestrol; (iii) lactams having from 5 to 6 annular members, which include the barbiturates, e.g., Phenobarbital and secobarbital, diphenylhydantoin, primidone, ethosuximide, and their metabolites; (iv) aminoalkylbenzenes, with alkyl of from 2 to 3 carbon atoms, which include the amphetamines; catecholamines, which include ephedrine, L-dopa, epinephrine; narceine; papaverine; and metabolites of the above; (v) benzheterocyclics which include oxazepam, chlorpromazine, tegretol, their derivatives and metabolites, the heterocyclic rings being azepines, diazepines and phenothiazines; (vi) purines, which includes theophylline, caffeine, their metabolites and derivatives; (vii) drugs derived from marijuana, which include cannabinol and tetrahydrocannabinol; (viii) hormones such as thyroxine, cortisol, triiodothyronine, testosterone, estradiol, estrone, progesterone, polypeptides such as angiotensin, LHRH, and immunosuppressants such as cyclosporin, FK506, mycophenolic acid (MPA), and so forth; (ix) vitamins such as A, B, e.g. B12, C, D, E and K, folic acid, thiamine; (x) prostaglandins, which differ by the degree and sites of hydroxylation and unsaturation; (xi) tricyclic antidepressants, which include imipramine, dismethylimipramine, amitriptyline, nortriptyline, protriptyline, trimipramine, chlomipramine, doxepine, and desmethyldoxepin; (xii) anti-neoplastics, which include methotrexate; (xiii) antibiotics, which include penicillin, chloromycetin, actinomycetin, tetracycline, terramycin, the metabolites and derivatives; (xiv) nucleosides and nucleotides, which include ATP, NAD, FMN, adenosine, guanosine, thymidine, and cytidine with their appropriate sugar and phosphate substituents; (xv) miscellaneous individual drugs which include methadone, meprobamate, serotonin, meperidine, lidocaine, procainamide, acetylprocainamide, propranolol, griseofulvin, valproic acid, butyrophenones, antihistamines, chloramphenicol, anticholinergic drugs, such as atropine, their metabolites and derivatives; (xvi) metabolites related to diseased states include spermine, galactose, phenylpyruvic acid, and porphyrin Type 1; (xvii) aminoglycosides, such as gentamicin, kanamicin, tobramycin, and amikacin; and (xviii) pesticides such as polyhalogenated biphenyls, phosphate esters, thiophosphates, carbamates, polyhalogenated sulfenamides, their metabolites and derivatives.

Polyvalent analytes are normally poly(amino acids), i.e., polypeptides and proteins, polysaccharides, nucleic acids, and combinations thereof. Such combinations include components of bacteria, viruses, chromosomes, genes, mitochondria, nuclei, cell membranes and the like. For the most part, the polyepitopic ligand analytes will have a molecular weight of at least about 5,000, more usually at least about 10,000. In the poly(amino acid) category, the poly(amino acids) of interest will generally be from about 5,000 to 5,000,000 molecular weight, more usually from about 20,000 to 1,000,000 molecular weight; among the hormones of interest, the molecular weights will usually range from about 5,000 to 60,000 molecular weight.

A wide variety of proteins may be considered as to the family of proteins having similar structural features, proteins having particular biological functions, proteins related to specific microorganisms, particularly disease causing microorganisms, etc. Such proteins include, for example, immunoglobulins, cytokines, enzymes, hormones, cancer antigens, nutritional markers, tissue specific antigens, etc. Such proteins include, by way of illustration and not limitation, protamines, histones, albumins, globulins, scleroproteins, phosphoproteins, mucoproteins, chromoproteins, lipoproteins, nucleoproteins, glycoproteins, T-cell receptors, proteoglycans, HLA, unclassified proteins, e.g., somatotropin, prolactin, insulin, pepsin, proteins found in human plasma, blood clotting factors, protein hormones such as, e.g., follicle-stimulating hormone, luteinizing hormone, luteotropin, prolactin, chorionic gonadotropin, tissue hormones, cytokines, cancer antigens such as, e.g., PSA, CEA, a-fetoprotein, acid phosphatase, CA19.9 and CA125, tissue specific antigens, such as, e.g., alkaline phosphatase, myoglobin, CPK-MB and calcitonin, and peptide hormones. Other polymeric materials of interest are mucopolysaccharides and polysaccharides.

For receptor analytes, the molecular weights will generally range from about 10,000 to about 2×10⁸, more usually from about 10,000 to about 10⁶. For immunoglobulins, IgA, IgG, IgE and IgM, the molecular weights will generally vary from about 160,000 to about 10⁶. Enzymes will normally range from about 10,000 to about 1,000,000 in molecular weight. Natural receptors vary widely, generally being at least about 25,000 molecular weight and may be about 10⁶ or higher molecular weight, including such materials as avidin, DNA, RNA, thyroxine binding globulin, thyroxine binding prealbumin, transcortin, etc.

The term analyte further includes oligonucleotide and polynucleotide analytes such as m-RNA, r-RNA, t-RNA, DNA, DNA-RNA duplexes, etc.

The analyte may be a molecule found directly in a sample such as biological tissue, including body fluids, from a host. The sample can be examined directly or may be pretreated to render the analyte more readily detectable by removing unwanted materials. The sample may be pretreated to separate or lyse cells; precipitate, hydrolyse or denature proteins; hydrolyze lipids; solubilize the analyte; or the like. Such pretreatment may include, without limitation: centrifugation; treatment of the sample with an organic solvent, for example, an alcohol, such as methanol; and treatment with detergents. The sample can be prepared in any convenient medium that does not interfere with an assay. An aqueous medium is preferred.

The analyte of interest may be determined by detecting an agent probative of the analyte of interest such as a specific binding pair member complementary to the analyte of interest, whose presence will be detected only when the analyte of interest is present in a sample. Thus, the agent probative of the analyte becomes the analyte that is detected in an assay.

Polynucleotide—a compound or composition which is a polymeric nucleotide having in the natural state about 50 to 500,000 or more nucleotides and having in the isolated state about 15 to 50,000 or more nucleotides, usually about 15 to 20,000 nucleotides, more frequently 15 to 10,000 nucleotides. Polynucleotide includes nucleic acids from any source in purified or unpurified form, naturally occurring or synthetically produced, including DNA (dsDNA and ssDNA) and RNA, usually DNA, and may be t-RNA, m-RNA, r-RNA, mitochondrial DNA and RNA, chloroplast DNA and RNA, DNA-RNA hybrids, or mixtures thereof, genes, chromosomes, plasmids, the genomes of biological material such as microorganisms, e.g., bacteria, yeasts, viruses, viroids, molds, fungi, plants, animals, humans, and fragments thereof, and the like.

Ligand—any organic compound for which a receptor naturally exists or can be prepared.

Hapten—a compound capable of binding specifically to corresponding antibodies, but does not itself act as an immunogen (or antigen) for preparation of the antibodies. Antibodies that recognize a hapten can be prepared against compounds comprised of the hapten linked to an immunogenic (or antigenic) carrier. Haptens are a subset of ligands.

Ligand analog—a modified ligand, an organic radical or analyte analog, usually of a molecular weight greater than 100, which can compete with the analogous ligand for a receptor, the modification providing means to join a ligand analog to another molecule. The ligand analog will usually differ from the ligand by more than replacement of a hydrogen with a bond which links the ligand analog to a hub or label, but need not. The ligand analog can bind to the receptor in a manner similar to the ligand. The analog could be, for example, an antibody directed against the idiotype of an antibody to the ligand.

Receptor (“antiligand”)—any compound or composition capable of recognizing a particular spatial and polar organization of a molecule, e.g., epitopic or determinant site. Illustrative receptors include naturally occurring receptors, e.g., thyroxine binding globulin, antibodies, enzymes, Fab fragments, lectins, nucleic acids, protein A, complement component C1q, and the like.

Substituted—means that a hydrogen atom of a molecule has been replaced by another atom, which may be a single atom such as a halogen, etc., or part of a group of atoms forming a functionality as described above. Such substituent may be a group or functionality imparting hydrophilicity. As discussed above, hydrophilicity may be achieved by a functional group having one or more atoms such as oxygen, nitrogen, sulfur, phosphorus, and so forth; such groups include sulfonate, sulfate, phosphate, amidine, phosphonate, carboxylate, hydroxyl particularly polyols, amine, ether, amide, and the like.

Signal producing system (“sps”)—one or more components, at least one component being a detectable label, which generate a detectable signal that relates to the amount of bound and/or unbound label, i.e. the amount of label bound or not bound to the compound being detected. The label is any molecule that produces or can be induced to produce a signal, and may be, for example, a fluorescer, radio-label, enzyme, chemiluminescer or photosensitizer. Thus, the signal is detected and/or measured by detecting enzyme activity, luminescence, light absorbance or radioactivity as the case may be.

Suitable labels include, by way of illustration and not limitation, enzymes such as alkaline phosphatase, glucose-6-phosphate dehydrogenase (“G6PDH”) and horseradish peroxidase; ribozyme; a substrate for a replicase such as QB replicase; promoters; dyes; fluorescers, such as fluorescein, isothiocyanate, rhodamine compounds, phycoerythrin, phycocyanin, allophycocyanin, o-phthaldehyde, and fluorescamine; complexes such as those prepared from CdSe and ZnS present in semiconductor nanocrystals known as Quantum dots; chemiluminescers such as isoluminol; sensitizers; coenzymes; enzyme substrates; radiolabels such as ¹²⁵I, ¹³¹I, ¹⁴C, ³H, ⁵⁷Co and ⁷⁵Se; particles such as latex or carbon particles; metal sol; crystallite; liposomes; cells, etc., which may be further labeled with a dye, catalyst or other detectable group. Suitable enzymes and coenzymes are disclosed in Litman, et al., U.S. Pat. No. 4,275,149, columns 19-28, and Boguslaski, et al., U.S. Pat. No. 4,318,980, columns 10-14; suitable fluorescers and chemiluminescers are disclosed in Litman, et al., U.S. Pat. No. 4,275,149, at columns 30 and 31; which are incorporated herein by reference.

There are numerous methods by which the label can produce a signal detectable by external means, desirably by visual examination, for example, by electromagnetic radiation, heat, and chemical reagents. The label or other sps members can also be bound to an sbp member, another molecule or to a support.

Labels include groups detectable by means of electromagnetic radiation or by electrochemical detection including dyes, fluorescers, chemiluminescers, and radioactive isotopes.

The label can directly produce a signal and, therefore, additional components are not required to produce a signal. Numerous organic molecules, for example fluorescers, are able to absorb ultraviolet and visible light, where the light absorption transfers energy to these molecules and elevates them to an excited energy state. This absorbed energy is then dissipated by emission of light at a second wavelength. Other labels that directly produce a signal include radioactive isotopes and dyes.

Alternately, the label may need other components to produce a signal, and the signal producing system would then include all the components required to produce a measurable signal, which may include substrates, coenzymes, enhancers, additional enzymes, substances that react with enzymic products, catalysts, activators, cofactors, inhibitors, scavengers, metal ions, and a specific binding substance required for binding of signal generating substances. A detailed discussion of suitable signal producing systems can be found in Ullman, et al., U.S. Pat. No. 5,185,243, columns 11-13, incorporated herein by reference.

The label and/or other sps member may be bound to an sbp member or to a support. For example, the label can be bound covalently to an sbp member such as, for example, an antibody; a receptor for an antibody, a receptor that is capable of binding to a small molecule conjugated to an antibody, or a ligand analog. Bonding of the label to the sbp member may be accomplished by chemical reactions that result in replacing a hydrogen atom of the label with a bond to the sbp member or may include a linking group between the label and the sbp member. Other sps members may also be bound covalently to sbp members. For example, two sps members such as a fluorescer and quencher can each be bound to a different antibody that forms a specific complex with the analyte. Formation of the complex brings the fluorescer and quencher in close proximity, thus permitting the quencher to interact with the fluorescer to produce a signal. Methods of conjugation are well known in the art. See, for example, Rubenstein, et al., U.S. Pat. No. 3,817,837, incorporated herein by reference.

Assay—method for the determination of the presence or amount of an analyte.

Sample—the material suspected of containing an analyte. Such samples, preferably from humans or animals, include biological fluids such as whole blood, serum, plasma, sputum, lymphatic fluid, semen, vaginal mucus, feces, urine, spinal fluid, saliva, stool, cerebral spinal fluid, tears, mucus, and the like; biological tissue such as hair, skin, sections or excised tissues from organs or other body parts; and so forth. Other samples include cell cultures and the like, plants, food, forensic samples such as paper, fabrics and scrapings, water, sewage, medicinals, etc. When necessary, the sample may be pretreated with reagents to liquefy the sample and release the analyte from binding substances. In many instances, the sample is plasma or serum.

Measuring the amount of an analyte—quantitative, semiquantitative, and qualitative methods as well as all other methods for determining an analyte are considered to be methods of measuring the amount of an analyte. For example, a method, which merely detects the presence or absence of an analyte in a sample suspected of containing the analyte, is considered to be included within the scope of the present invention. The terms “detecting” and “determining,” as well as other common synonyms for measuring, are contemplated within the scope of the present invention.

Ancillary Materials—Various ancillary materials will frequently be employed in the assay in accordance with the present invention. For example, buffers will normally be present in the assay medium, as well as stabilizers for the assay medium and the assay components. Frequently, in addition to these additives, proteins may be included, such as albumins; organic solvents such as formamide; quaternary ammonium salts; polyanions such as dextran sulfate; surfactants, particularly non-ionic surfactants; binding enhancers, e.g., polyalkylene glycols; or the like.

Wholly or partially sequentially—when various agents are combined other than concomitantly (simultaneously), one or more may be combined with one or more of the remaining agents to form a subcombination.

A method for the quantitative and/or qualitative determination of an analyte an assay for determining the presence or amount of an analyte. Quantitative, semiquantitative, and qualitative methods as well as all other methods for determining an analyte are considered to be methods of measuring the amount of an analyte. For example, a method which merely detects the presence or absence of an analyte in a sample suspected of containing the analyte is considered to be included within the scope of the present invention. The terms “detecting” and “determining,” as well as other common synonyms for measuring, are contemplated within the scope of the present invention.

In the most common assays for the quantitative and/or qualitative determination of an analyte, the analyte is bound by a specific binding partner, preferably by a specific binding partner associated with a solid support and/or a component of a signal producing system, such as a label or a reporter molecule. Said specific binding partner may be bound directly, e.g. covalently or by adsorption, or indirectly to the solid support and/or to the component of a signal producing system. Indirect binding refers to the spatial association of two specific binding partners that are not members of a specific binding pair through a series of bonds between different binding pairs. Exemplary of indirect binding is the indirect binding of a biotinylated antibody to a label upon the binding of said biotinylated antibody to avidin attached to the label. A further example is the indirect binding of IgM to a solid support upon the binding of IgM to anti-IgM-antibodies attached to the solid support.

Another aspect of the present invention relates to kits useful for conveniently performing an assay for the determination of an analyte. In one embodiment a kit comprises in packaged combination an antibody for the analyte in accordance with embodiments of the invention and other reagents for conducing the assay such as, for example, an enzyme conjugate and the like. In another embodiment a kit of the invention comprises in packaged combination an antibody bound to a support and pretreated in accordance with an embodiment of the present methods, a conjugate of a label and a second antibody, which may or may not have been pretreated as described herein. Other kit embodiments are also included and the reagents in the kit depend upon the particular assay format. However, at least one of the reagents comprises an antibody reagent that has been pretreated in accordance with one embodiment of the present methods.

To enhance the versatility of the subject invention, the kit reagents can be provided in packaged combination, in the same or separate containers, in liquid or lyophilized form so that the ratio of the reagents provides for substantial optimization of the method and assay. The reagents may each be in separate containers or various reagents can be combined in one or more containers depending on the cross-reactivity and stability of the reagents.

The kit can further include other separately packaged reagents for conducting an assay such as additional sbp members, ancillary reagents such as an ancillary enzyme substrate, and so forth. The relative amounts of the various reagents in the kits can be varied widely to provide for concentrations of the reagents that substantially optimize the reactions that need to occur during the present method and further to optimize substantially the sensitivity of the assay. Under appropriate circumstances one or more of the reagents in the kit can be provided as a dry powder, usually lyophilized, including excipients, which on dissolution will provide for a reagent solution having the appropriate concentrations for performing a method or assay in accordance with the present invention. The kit can further include a written description of a method in accordance with the present invention as described above.

The invention is demonstrated further by the following illustrative examples.

### EXAMPLES

Parts and percentages herein are by weight unless otherwise indicated. Temperatures are in degrees Centigrade (° C.).

### Abbreviations:

### EDTA—ethylenediamine tetraacetic acid

### EGTA—ethylene glycol bis(2-aminoethyl ether)-N,N,N′N′-tetraacetic acid

### NSB—non-specific binding

hr—hour(s)

### BSA—bovine serum albumin

### PBS—phosphate buffered saline

### Materials:

### Chemicals:

Unless noted otherwise, all chemicals were purchased from the Sigma-Aldrich Company (St. Louis Mo.).

### Test Samples:

Calibrator, which contains 5% BSA and preservatives, Human plasma, serum and QCs, purchased from Bio-Rad Laboratories, Hercules Calif.

### Test Assays:

Dimension® NT-proBNP method

### Example 1

### Pretreatment of Conjugated Anti-NT-proBNP Antibody

The NT-proBNP method on the Dimension® clinical chemistry system is based on chrome sandwich immunoassay technology with cascade detection system. The immunoreagent formulations are: capture antibody-coated chrome particles (chrome particle reagent) and enzyme-label antibody conjugate. Due to the very low analyte concentrations, the NT-proPBNP method uses alkaline phosphatase as the enzyme label with the Rabin cascade detection system (Harbron, et al. Analytical Biochemistry 206, 119-124, 1992). Briefly, the Rabin cascade detection system operates as follows: Alkaline phosphatase (ALP; EC 3.1.3.1) conjugated to the assay antibody dephosphorylates flavin adenine dinucleotide-3′-phosphate (FADP) to produce cofactor flavin adenine dinucleotide (FAD), which binds stoichiometrically to inactive apo D-amino acid oxidase (D-MO). The resulting active holo D-MO oxidizes D-proline to produce hydrogen peroxide, which is quantified by the horseradish peroxidase-mediated conversion of 3,5-dichloro-2-hydroxybenzenesulfonic acid and 4-aminoantipyrine to a colored product, which can be measured bichromatically at 510 and 700 nanometers.

The capture antibody recognizes an epitope in the 1-21 amino acid region of the peptide, while the labeled antibody recognizes an epitope in the 39-50 region. The capture antibody is conjugated to the chromium dioxide surface by means of its amino groups onto the glutaraldehyde-activated chromium dioxide surface. The procedure is discussed in more detail in U.S. Pat. No. 4,661,408, issued to Lau, et al., Apr. 28, 1987, the relevant disclosure of which is incorporated herein by reference.

Utilization of both enzyme labeled polyclonal antibody and capture antibody to develop an assay for NT-proBNP showed significant lot-to-lot variation in non-specific binding. Non-specific binding (NSB) caused the elevation of the assay background signal and consumed enzyme substrate even in the absence of analyte. Removing or reducing NSB was achieved in accordance with the present methods as discussed below.

In this example, a 2.5 mL suspension of antibody-coupled chrome particles (chrome particle reagent) was exchanged into 2.5 mL 100 mM NaH2PO4 buffer containing 5 mM EDTA, pH 6.0. The chrome particle concentration was 5% solids. The 2.5 mL chrome particle reagent suspension (pH 6.0) was mixed with 0.278 mL of a mixture of dithiothreitol (0.074 M in water) and 2-mercaptoethanol (0.22 M in water). The chrome particle reagent slurry was incubated on a rocker for 5 hrs at 37° C. After incubation, the particles of the chrome particle reagent slurry were exchanged into 2.5 mL 10 mM phosphate buffer (pH 7.0).

The 2.5 mL suspension of antibody-coupled chrome particles was divided into five 0.5 mL aliquots and treated using different conditions (A-E below):
A. Incubated with 0.1 M citrate, pH 2.90 at 25° C. and rocked for 3 hours;
B. Incubated with 0.1 M citrate, pH 2.90 at 25° C. and rocked for 5 minutes;
C. Incubated with 0.1M citrate, 2M MgCl₂, pH 1.95 at 25° C. and rocked for five minutes;
D. Incubated with 2M MgCl₂ at 25° C. and rocked for 3 hours
E. No additional treatment

Following the individual treatments outlined above, each chrome particle reagent slurry mixture was washed 3 times with a high salt buffer (2M MgCl₂). Each chrome particle reagent slurry mixture was then washed 3 times with 10 mM phosphate buffer and then exchanged into 0.5 mL 10 mM phosphate buffer (pH 7.0) so that the concentration of each aliquot would remain at 5% solids.

After the chrome particle reagent slurries were exchanged into 10 mM phosphate buffer (pH 7.0), 0.4 mL (0.8 mL×batch size) 30% BSA were added and the chrome particle reagent slurries were incubated for 4 hours at 45° C. (with rocking). Upon completion of the incubation with BSA, 0.9 mL 2M glycine was added to the reaction mixture and the suspension of chrome particle reagent was incubated (and rocked) for 2 hours at room temperature (25° C.).

The final chrome particle reagent slurries were washed with 10 mM phosphate buffer (pH 7.0) and exchanged into 10 mM phosphate buffer (pH 7.0) for testing. Chrome particle reagent slurries were stored at 2-8° C.

After the incubations as described in A-E above, each treated aliquot was mixed with the antibody-alkaline phosphatase conjugate to detect NSB. In the absence of analyte, lower signals in milli-absorbance unit (mAU) indicate lower bridging of capture antibody and labeled antibody via non-specific binding interference substances. The results are shown in Table 1.

TABLE 1

| Treatment | mAUs |
|---|---|
| A | 32 |
| B | 52 |
| C | 56 |
| D | 156 |
| E | 149 |

### Example 2

### Pretreatment of Anti-NT-proBNP Antibody

A 4 mL solution, containing 56 mg protein, of anti-NTproBNP antibody in 0.1M sodium phosphate-5.0 mM EDTA, pH 6.0 (pH 6.0 buffer) was mixed with 0.44 mL of a 0.1 M solution of dithiothreitol in the pH 6.0 buffer. After heating the mixture at 37° C. for 1 hr, the protein mixture was passed through a SEPHADEX® G25 column (2.6×30 cm) equilibrated and eluted in 10 mM sodium phosphate-300 mM NaCl, pH 7.0 (PBS). Protein-containing fractions were combined to provide 48.3 mg of the reduced antibody. The reduced antibody was titrated to contain 12.3 moles of free thiols per mole of the protein. A 7.1 mL solution of the reduced antibody, containing 46 mg protein, was combined with 0.71 mL of a 1.0 M sodium phosphate and then titrated by a slow addition of a 2.0 M solution of citric acid so that final pH of the reaction mixture was 2.5. A 0.85 mL of the citric acid solution was required for this purpose. The reaction mixture was incubated at 4° C. for 3 hrs and then adjusted to a pH 7.0 by a slow addition of 2.3 mL of 2.0 M NaOH. The protein solution was centrifuged at 3000 rpm for 15 minutes and clear solution thus obtained was found to contain 45 mg antibody.

Immobilization of the anti-NTproBNP antibody, pretreated above in accordance with the present invention, on the surface of chrome particles: A 2 mL suspension of chrome particles (5% solid) was mixed with 1.2 mL of 10 mM sodium phosphate-300 mM NaCl, pH 7.0 (PBS) containing 0.8 mL of 25% glutaraldehyde. After reacting for 3 hrs the chrome particles were separated from excess glutaraldehyde by repeated washing with PBS and then mixed with 4 mL of the reduced-acid treated anti-NTproBNP antibody solution (as prepared above) containing 1.5 mg/mL protein. After 16 hrs at 4° C., reaction mixture was combined with 1.6 mL 30% BSA and then after 4 hrs mixed with 5.6 mL of 2.0 M Glycine. The chrome particles were washed with 10 mM sodium phosphate, pH 7.0, and stored suspended in 2 mL of 10 mM sodium phosphate, pH 7.0 at 4° C. In one case, the low pH treatment as described above was carried out in the presence of 0.04% TWEEN 20® surfactant.

The above pretreated antibody used in the preparation of the chrome particle reagent successfully lowered NSB as shown in Table 2 when the chrome particle reagent was employed in assays as described above in Example 1.

TABLE 2

| Treatment | mAUs |
|---|---|
| pH 2.5 + DTT | 8 |
| pH 2.5 + DTT/Tween 20 | 10 |
| No treatment | 237 |

### Example 3

### Pretreatment of Anti-NT-proBNP Antibody in the Presence and Absence of Thiol-Containing Agents

A 2 mL solution of anti-NTproBNP antibody (containing 28 mg protein) in 0.1M sodium phosphate-5.0 mM EDTA, pH 6.0 (pH 6.0 buffer) was buffer exchanged with a solution containing 100 mM citrate, 1.0 M sodium chloride, pH 2.5. The antibody was incubated in the buffer at pH 2.5 for about 1 hour at 25° C. After the low pH treatment, the antibody solution was passed through a SEPHADEX® G25 column (2.6×30 cm), equilibrated and eluted in 10 mM sodium phosphate-300 mM NaCl, pH 7.0 (PBS). Protein-containing fractions were combined and diluted in 0.1M sodium phosphate-5.0 mM EDTA, pH 6.0 to contain 3 mg/mL protein. In one experiment, the low pH treatment as described above was carried out in the presence of 0.074 M dithiothreitol (DTT).

The above pretreated antibody preparations were conjugated to chrome particles to prepare chrome reagent using a procedure similar to that described above. The use of the pretreated antibody in the preparation of the chrome particle reagent successfully lowered NSB as shown in Table 3 when the chrome particle reagent was employed in assays as described above in Example 1.

TABLE 3

| Treatment | mAUs |
|---|---|
| pH 2.5 | 35 |
| pH 2.5 + DTT | 16 |
| No treatment | 237 |

### Example 4

### Pretreatment of Capture Ab-Chrome Using Thiol-Containing Agents

A 2 mL suspension of the antibody-coupled chrome particles (prepared essentially as described above) was exchanged with 0.1M sodium phosphate-5.0 mM EDTA, pH 6.0. The particles were suspended in 2 mL of the pH 6.0 buffer and mixed with 0.23 mL of a mixture of dithiothreitol (0.074 M of the pH 6.0 buffer) and 2-mercaptoethanol (0.22 M of the pH 6.0 buffer). After treatment for 2 hrs at 37° C., the particles were exchanged with PBS and treated with 0.8 mL of 30% BSA and then with 2.8 mL of 2.0 M glycine essentially as described above. The particles were then washed and stored in PBS at 4° C. as described above. The chrome particle reagent prepared with the antibody pretreated as described above exhibited a reduction in NSB as shown in Table 4 when the chrome particle reagent was employed in assays as described above in Example 1.

TABLE 4

| Treatment | mAUs |
|---|---|
| +Thiol agents | 149 |
| −Thiol agents | 237 |

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

What is claimed is:

1. A method for preparing an antibody reagent for use in an immunoassay, the method comprising treating the antibody reagent at a pH of about 2.0 to about 3.5 to reduce non-specific binding of the antibody reagent with the proviso that, when the antibody reagent is unconjugated antibody, the treating is carried out in the absence of a chromatographic material.

2. A method according to claim 1 further comprising treating the antibody reagent with a reducing agent in an amount sufficient to reduce non-specific binding of the antibody reagent.

3. A method according to claim 1 wherein the antibody reagent is an unconjugated antibody or an antibody conjugated to a support, a member of a signal producing system or a member of a specific binding pair.

4. A method according to claim 2 wherein the reducing agent is a thiol-containing reducing agent or a borohydride or a phosphine.

5. A method according to claim 2 wherein the reducing agent is a single reducing agent or a combination of two or more thiol-containing reducing agents.

6. A method according to claim 2 wherein the reducing agent is dithiothreitol or β-mercaptoethanol or a combination thereof.

7. A method according to claim 1 wherein the pH is about 2.5 to about 3.5.

8. A method for determining the presence and/or amount of an analyte in a sample suspected of containing the analyte, the method comprising:
(a) providing in combination the sample and reagents for detecting the analyte wherein at least one of the reagents comprises an antibody reagent prepared according to the method of claim 1,
(b) incubating the combination under conditions for binding of the analyte to one or more of the reagents, and
(c) detecting the presence and/or amount of binding of the analyte to one or more of the reagents, the presence and/or amount of the binding being related to the presence and/or amount of the analyte in the sample.

9. A method according to claim 8 wherein the antibody reagent is an antibody conjugated to a support.

10. A method according to claim 9 wherein the solid support comprises particles.

11. A method according to claim 8 wherein the antibody reagent comprises a member of a signal producing system.

12. A method according to claim 8 wherein the at least one other of the reagents comprises a second antibody specific for the analyte.

13. A method according to claim 12 wherein the second antibody is pretreated at a pH of about 2.0 to about 3.5 and optionally with a reducing agent in an amount sufficient to reduce non-specific binding of the antibody reagent

14. A method for determining the presence and/or amount of an analyte in a sample suspected of containing the analyte, the method comprising:
(a) providing in combination the sample and reagents for detecting the analyte wherein at least one of the reagents comprises an antibody reagent prepared according to the method of claim 2,
(b) incubating the combination under conditions for binding of the analyte to one or more of the reagents, and
(c) detecting the presence and/or amount of binding of the analyte to one or more of the reagents, the presence and/or amount of the binding being related to the presence and/or amount of the analyte in the sample.

15. A method according to claim 14 wherein the antibody reagent is an antibody conjugated to a support.

16. A method according to claim 15 wherein the solid support comprises particles.

17. A method according to claim 14 wherein the antibody reagent comprises a member of a signal producing system.

18. A method according to claim 14 wherein the at least one other of the reagents comprises a second antibody specific for the analyte.

19. An antibody reagent prepared according to the method of claim 1.

20. An antibody reagent according to claim 19 wherein the antibody reagent is an unconjugated antibody or an antibody conjugated to a support, to a member of a signal producing system or to a member of a specific binding pair.

21. An antibody reagent according to claim 20 wherein the antibody reagent is an antibody conjugated to a solid support wherein the solid support comprises particles.

22. A method for preparing an antibody reagent for use in an immunoassay, the method comprising:
(a) treating the antibody reagent with a reducing agent in an amount sufficient to reduce non-specific binding of the antibody reagent and
(b) optionally treating the antibody reagent at a pH of about 2.0 to about 3.5.

23. A method according to claim 22 wherein the antibody reagent is an unconjugated antibody or an antibody conjugated to a support, a member of a signal producing system or a member of a specific binding pair.

24. A method according to claim 22 wherein the reducing agent is a thiol-containing reducing agent.

25. A method according to claim 22 wherein the reducing agent is a combination of two or more thiol-containing reducing agents.

26. A method according to claim 25 wherein the reducing agent is a combination of dithiothreitol and β-mercaptoethanol.

27. A method according to claim 22 wherein the pH is about 2.5 to about 3.5.

28. A method for preparing an antibody reagent for use in an immunoassay, the method comprising:
(a) treating the antibody reagent at a pH of about 2.0 to about 3.5. and
(b) treating the antibody reagent with a reducing agent in an amount sufficient to reduce non-specific binding of the antibody reagent
